(19) United States
(12) Patent Application Publication
(10) Pub. No.: US 2015/0183703 A1
(43) Pub. Date: Jul. 2, 2015

(54) SYNTHESIS OF DIACIDS

(71) Applicants: Pui Kwan Wong, Singapore (SG); Chuanzhao Li, Singapore (SG); Ludger Stubbs, Singapore (SG); Martin Van Meurs, Singapore (SG); Daniel Gait Anak Kumbang, Singapore (SG); Sharon Chun Yan Lim, Singapore (SG); Eite Drent, Singapore (SG)

(72) Inventors: Pui Kwan Wong, Singapore (SG); Chuanzhao Li, Singapore (SG); Ludger Stubbs, Singapore (SG); Martin Van Meurs, Singapore (SG); Daniel Gait Anak Kumbang, Singapore (SG); Sharon Chun Yan Lim, Singapore (SG); Eite Drent, Singapore (SG)

(73) Assignee: Agency for Science, Technology and Research, Singapore (SG)

(21) Appl. No.: 14/007,169

(22) PCT Filed: Mar. 28, 2012
(86) PCT No.: PCT/SG2012/000107

§ 371 Date: Jan. 7, 2014

Related U.S. Application Data
(60) Provisional application No. 61/468,494, filed on Mar. 28, 2011.

(51) Int. Cl. C07C 51/14; C07D 307/33; C08G 69/28; C07C 51/00; C08H 8/00; C07C 51/09; C07C 51/42

(52) U.S. Cl. CPC C07C 51/14; C07C 51/09; C07D 307/33; C07C 51/42; C07C 51/00; C08H 8/00; C08G 69/28

(57) ABSTRACT

The invention relates to a process for preparing a dicarboxylic acid comprising the steps: (a) heating a lactone in the presence of a first catalyst system to produce an alkenoic acid; and (b) contacting the alkenoic acid with carbon monoxide, water and a second catalyst system to produce a reaction composition comprising the second catalyst system and the dicarboxylic acid.

### RELATED APPLICATION

This application claims priority from U.S. Provisional Patent Application No. 61/468,494, which is incorporated herein in its entirety by cross-reference.

### TECHNICAL FIELD

The present invention relates to processes for synthesising dicarboxylic acids.

### BACKGROUND OF THE INVENTION

Any discussion of the background art throughout the specification should in no way be considered as an admission that such background art is prior art, nor that such background art is widely known or forms part of the common general knowledge in the field.

Dicarboxylic acids may be used as monomer units in the preparation of polymers. For example, adipic acid is used as a monomer unit in the preparation of Nylon 6-6.

Existing processes for producing many diacids rely on fossil feedstocks. Among commercial polymers, processes for preparing Nylons and Nylon monomers require a high use of energy and chemicals. Concerns for the future availability of fossil feedstocks and environmental factors have led to significant interest in chemicals and polymers derived from renewable sources, such as biomass.

Accordingly, there is a need to provide processes for preparing diacids, such as adipic acid, from renewable feedstock.

### SUMMARY OF INVENTION

According to a first aspect of the present invention there is provided a process for preparing a dicarboxylic acid comprising the steps of:
(a) heating a lactone in the presence of a first catalyst system to produce an alkenoic acid; and
(b) contacting the alkenoic acid with carbon monoxide, water and a second catalyst system to produce a reaction composition comprising the second catalyst system and the dicarboxylic acid.

The following options may be used in combination with the first aspect, either individually or in any suitable combination.

Step (a) may be carried out substantially in the absence of water. The process according to the first aspect above may further comprise a step of removing water from the lactone and/or from the first catalyst system prior to step (a).

The heating of the lactone in step (a) may comprise reactive distillation, thereby providing a distillate comprising the alkenoic acid.

Step (a) may be carried out at a temperature at or above the normal boiling point of the lactone. Step (a) may be carried out at a temperature of between about 150° C. and about 370° C. Step (a) may be carried out at a pressure of between about 0.5 bar and 30 bar. Step (a) may be carried out at a pressure of about 1 bar.

The first catalyst system may comprise an acidic catalyst. The first catalyst system may comprise a heterogeneous solid catalyst. The first catalyst system may comprise a homogeneous catalyst. The first catalyst system may comprise one or more of alumina, silica, a zeolite, a clay, sulphuric acid, p-toluenesulfonic acid and methanesulfonic acid. The first catalyst system may, for example, comprise a mixture of alumina and silica.

The first catalyst system and the temperature and pressure of step (a) may be such that conversion of lactone to alkenoic acid in step (a) is greater than about 95%.

The alkenoic acid produced in step (a) may comprise a plurality of isomers.

Step (b) may be carried out substantially in the absence of oxygen. Step (b) may be carried out at a temperature of between about 50° C. and about 150° C. Step (b) may be carried out at a temperature of between about 80° C. and about 120° C. Step (b) may be carried out at a pressure of between about 1 bar and about 150 bar. Step (b) may be carried out at a pressure of between about 3 bar and about 80 bar. Step (b) may be carried out at a pressure of between about 5 bar and about 60 bar.

The second catalyst system may comprise a palladium catalyst. The palladium catalyst may have the formula (I):

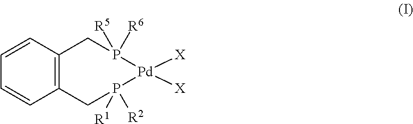

wherein X is a ligand and each of R¹, R², R⁵ and R⁶ is independently an optionally substituted organic group or R¹ and R² and/or R⁵ and R⁶, together with the P atom to which they are attached, form a cyclic group.

In step (b), the second catalyst system may be prepared in situ. The process according to the first aspect above may further comprise the step of preparing the palladium catalyst. The palladium catalyst may be prepared by combining a palladium compound, a bidentate diphosphine and an acid.

In formula (I) above, X may be derived from the acid. The bidentate diphosphine may have the formula (II):

(R¹)(R²)P—R³—Ar—R⁴—P(R⁵)(R⁶) (II)

wherein
Ar is an optionally substituted aromatic group;
R¹ and R² are either the same or different and represent a tertiary alkyl or, together with the P atom to which they are attached, form a phospha trioxa-adamantane group having formula (III):

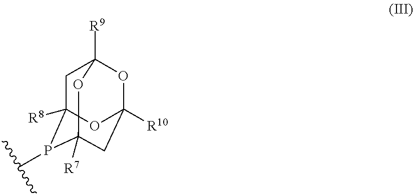

wherein R⁷, R⁸, R⁹ and R¹⁰ each independently represent an optionally substituted hydrocarbyl group;
R³ and R⁴ each independently represent an optionally substituted alkylene group; and
R⁵ and R⁶ each independently represent an optionally substituted organic group or together with the P atom to which they are attached, form a cyclic group.

In formula (I) and formula (II), R⁵ and R⁶ may each independently represent a tertiary alkyl, cycloalkyl, heterocyclyl or aryl. In formula (I) and formula (II), R⁵ and R⁶ may each independently represent tertiary butyl, adamantyl or phenyl, or, together with the P atom to which they are attached, form a phospha trioxa-adamantane group of formula (III).

The palladium compound from which the second catalyst is prepared may be selected from the group consisting of palladium carboxylates and palladium(0) compounds. The palladium compound may be palladium acetate, tris(dibenzylideneacetone)dipalladium(0) or palladium acetylacetonate.

The bidentate diphosphine may be 1,2-bis[di(t-butyl)phosphinomethyl]benzene.

The acid used in preparing the palladium catalyst may have a pKa of less than about 5 (measured in water at 18° C.). The acid may be selected from the group consisting of sulfonic acids, sulphuric acids, phosphorous acids and carboxylic acids. The acid may, for example, be a C₁-C₁₀ aliphatic acid. The acid may be selected from the group consisting of methanesulfonic acid, triflic acid, trifluoroacetic acid and acetic acid. The acid may be the alkenoic acid obtained in step (a). The acid may be present in molar excess relative to the palladium compound. The molar ratio of the acid to the palladium compound may be from 2 to 10000 (i.e. 2:1 to 10000:1). The molar ratio of the acid to the palladium compound may be from 5 to 5000 (i.e. 5:1 to 5000:1). The molar ratio of the acid to the palladium compound may be from 5 to 300 (i.e. 5:1 to 300:1).

The palladium catalyst may be prepared by combining palladium acetate, 1,2-bis[di(t-butyl)phosphinomethyl]benzene, and methane sulfonic acid.

The second catalyst system and the temperature and pressure of step (b) may be such that the conversion of alkenoic acid to dicarboxylic acid in step (b) is greater than about 95%.

Step (b) may be carried out in a solvent. The solvent may be such that the dicarboxylic acid can be separated from the unreacted alkenoic acid by reducing the temperature of the reaction composition. The solvent may be the lactone used in step (a).

A portion of the lactone may be unreacted after step (a). In this case, the process according to the first aspect above may further comprise step (a1) of separating part or substantially all the unreacted lactone from the pentenoic acid. The process according to the first aspect above may further comprise step (a2) of recycling the separated unreacted lactone to step (a). The process according to the first aspect above may further comprise the steps of: (a1) separating part or substantially all of the unreacted lactone from the alkenoic acid; and (a2) recycling part or substantially all of the separated unreacted lactone from step (a1) to step (a).

The reaction composition produced by step (b) may comprise part or substantially all of the unreacted lactone from step (a). In this case, the process according to the first aspect above may comprise a step (b1) of separating part or substantially all the unreacted lactone from the dicarboxylic acid. The process according to the first aspect above may comprise a step (b2) of recycling the separated unreacted lactone to step (a). The process according to the first aspect above may comprise the steps of: (b1) separating part or substantially all of the unreacted lactone from the dicarboxylic acid; and (b2) recycling part or substantially all of the separated unreacted lactone from step (b1) to step (a).

The reaction composition produced by step (b) may comprise unreacted alkenoic acid. In this case, the process according to the first aspect above may comprise a step (b3) of separating part or substantially all the unreacted alkenoic acid from the dicarboxylic acid. The process according to the first aspect above may comprise a step (b4) of recycling the separated unreacted alkenoic acid to step (a). The process according to the first aspect above may comprise the steps of: (b3) separating part or substantially all of the unreacted alkenoic acid from the dicarboxylic acid and (b4) recycling part or substantially all of the separated unreacted alkenoic acid from step (b3) to step (a) and/or recycling part or substantially all of the separated unreacted alkenoic acid from step (b3) to step (b).

The process according to the first aspect may further comprise the step of: (c) separating the dicarboxylic acid from the remainder of the reaction composition to produce a first portion comprising the dicarboxylic acid and a second portion comprising the second catalyst system. The first portion may comprise substantially none of the second catalyst system. Step (c) may comprise reducing the temperature of the reaction composition such that the dicarboxylic acid crystallises. The process according to the first aspect above may further comprise the step of washing the first portion. The process according to the first aspect above may further comprise the step of: (d) recycling the second portion, comprising the second catalyst system, to step (b). The process according to the first aspect above may comprise the steps of: (c) separating the dicarboxylic acid from the remainder of the reaction composition of step (b) to produce a first portion comprising the dicarboxylic acid and a second portion comprising the second catalyst system; and (d) recycling the second portion, comprising the second catalyst system, to step (b).

The lactone used in step (a) may be γ-valerolactone, whereby the alkenoic acid is pentenoic acid and the dicarboxylic acid is adipic acid. The pentenoic acid may comprise one or more, or optionally all, of 2-pentenoic acid, 3-pentenoic acid and 4-pentenoic acid. The process according to the first aspect above may further comprise the step of preparing the γ-valerolactone by hydrogenation of levulinic acid. The process according to the first aspect above may further comprise the step of preparing the levulinic acid by acid catalysed hydrolysis of cellulose. The process according to the first aspect above may further comprise the step of preparing the cellulose by cracking lignocellulose.

The process according to the first aspect above may comprise the steps: (a) heating a lactone in the presence of a first catalyst system to produce an alkenoic acid; and (b) contacting the alkenoic acid with carbon monoxide, water and a second catalyst system to produce a reaction composition comprising the second catalyst system and the dicarboxylic acid, wherein the heating of the lactone in step (a) comprises reactive distillation, thereby providing a distillate comprising the alkenoic acid.

In an embodiment there is provided a process for preparing a dicarboxylic acid comprising the steps:
(a) heating a lactone in the presence of a first catalyst system to produce an alkenoic acid; and
(b) contacting the alkenoic acid with carbon monoxide, water and a second catalyst system to produce a reaction composition comprising the second catalyst system and the dicarboxylic acid,

wherein step (a) is carried out substantially in the absence of water.

In another embodiment there is provided a process for preparing a dicarboxylic acid comprising the steps:
(a) heating a lactone in the presence of a first catalyst system to produce an alkenoic acid; and
(b) contacting the alkenoic acid with carbon monoxide, water and a second catalyst system to produce a reaction composition comprising the second catalyst system and the dicarboxylic acid,
   wherein step (a) is carried out substantially in the absence of water and the heating of the lactone in step (a) comprises reactive distillation, thereby providing a distillate comprising the alkenoic acid.

In another embodiment there is provided a process for preparing a dicarboxylic acid comprising the steps:
(a) heating a lactone in the presence of a first catalyst system to produce an alkenoic acid, the first catalyst system comprising an acidic catalyst; and
(b) contacting the alkenoic acid with carbon monoxide, water and a second catalyst system, the second catalyst system comprising a palladium catalyst, to produce a reaction composition comprising the second catalyst system and the dicarboxylic acid,

wherein step (a) is carried out substantially in the absence of water and the heating of the lactone in step (a) comprises reactive distillation, thereby providing a distillate comprising the alkenoic acid.

In another embodiment there is provided a process for preparing a dicarboxylic acid comprising the steps:
(a) heating a lactone in the presence of a first catalyst system to produce an alkenoic acid, the first catalyst system comprising an acidic catalyst; and
(b) contacting the alkenoic acid with carbon monoxide, water and a second catalyst system, the second catalyst system comprising a palladium catalyst, to produce a reaction composition comprising the second catalyst system and the dicarboxylic acid,

wherein step (a) is carried out substantially in the absence of water and the heating of the lactone in step (a) comprises reactive distillation, thereby providing a distillate comprising the alkenoic acid, and

wherein the palladium catalyst is prepared by combining palladium acetate, 1,2-bis[di(t-butyl)phosphinomethyl]benzene, and methane sulfonic acid.

In another embodiment there is provided a process for preparing adipic acid comprising the steps:
(a) heating γ-valerolactone in the presence of a first catalyst system to produce pentenoic acid, the first catalyst system comprising an acidic catalyst; and
(b) contacting the pentenoic acid with carbon monoxide, water and a second catalyst system, the second catalyst system comprising a palladium catalyst, to produce a reaction composition comprising the second catalyst system and the adipic acid,

wherein step (a) is carried out substantially in the absence of water and the heating of the γ-valerolactone in step (a) comprises reactive distillation, thereby providing a distillate comprising the pentenoic acid, and

wherein the palladium catalyst is prepared by combining palladium acetate, 1,2-bis[di(t-butyl)phosphinomethyl]benzene, and methane sulfonic acid.

In another embodiment there is provided a process for preparing adipic acid comprising the steps:
(a) heating γ-valerolactone in the presence of a first catalyst system to produce pentenoic acid, the first catalyst system comprising a mixture of alumina and silica; and
(b) contacting the pentenoic acid with carbon monoxide, water and a second catalyst system, the second catalyst system comprising a palladium catalyst, to produce a reaction composition comprising the second catalyst system and the adipic acid,

wherein step (a) is carried out substantially in the absence of water and the heating of the γ-valerolactone in step (a) comprises reactive distillation, thereby providing a distillate comprising the pentenoic acid, and

wherein the palladium catalyst is prepared by combining palladium acetate, 1,2-bis[di(t-butyl)phosphinomethyl]benzene, and methane sulfonic acid.

In another embodiment there is provided a process for preparing adipic acid comprising the steps:
(a) heating γ-valerolactone to a temperature in the range 200 to 350° C., at a pressure of between about 1 to 40 bar, in the presence of a first catalyst system to produce pentenoic acid, the first catalyst system comprising a mixture of alumina and silica; and
(b) contacting the pentenoic acid with carbon monoxide, water and a second catalyst system, the second catalyst system comprising a palladium catalyst, at a temperature in the range 80 to 120° C. and a pressure in the range 1 to 80 bar, to produce a reaction composition comprising the second catalyst system and the adipic acid,

wherein step (a) is carried out substantially in the absence of water and the heating of the γ-valerolactone in step (a) comprises reactive distillation, thereby providing a distillate comprising the pentenoic acid, and

wherein the palladium catalyst is prepared by combining palladium acetate, 1,2-bis[di(t-butyl)phosphinomethyl]benzene, and methane sulfonic acid.

In another embodiment there is provided a process for preparing adipic acid comprising the steps:
(a) refluxing γ-valerolactone in the presence of a mixture of alumina and silica as a first catalyst system at a temperature in the range 200 to 350° C. and at a pressure of about 1 bar for 1 to 3 hours, substantially in the absence of water, to produce a composition comprising pentenoic acid;
(a′) distilling the composition from step (a) to produce a distillate comprising pentenoic acid;
(b) degassing the distillate and combining the degassed distillate with degassed deionised water and a degassed solvent (e.g., diglyme);
(b′) combining palladium acetate, 1,2-bis[di(t-butyl)phosphinomethyl]benzene and methane sulfonic acid to form a second catalyst system;
(b″) adding the second catalyst system to the combined distillate, water and solvent under a stream of argon gas and flushing with carbon monoxide;
(b′″) heating at a temperature in the range 80 to 120° C. and a pressure in the range 1 to 80 bar of carbon monoxide for 2 to 10 hours, to produce a reaction composition comprising the second catalyst system and the adipic acid,

According to a second aspect of the present invention there is provided a dicarboxylic acid prepared in accordance with the process of the first aspect above. The dicarboxylic acid may have a purity of greater than about 99%. The dicarboxylic acid may be adipic acid.

According to a third aspect of the present invention there is provided a method of preparing Nylon 6-6 comprising the step of copolymerising adipic acid prepared in accordance with the third aspect above with hexamethylenediamine, thereby forming the Nylon 6-6.

### BRIEF DESCRIPTION OF THE DRAWINGS

Embodiments of the present invention will now be described, by way of an example only, with reference to the accompanying drawings wherein:

FIG. 1 is a schematic diagram of an embodiment of the process for preparing a diacid according to the present invention.

### DETAILED DESCRIPTION OF THE PREFERRED EMBODIMENTS

Disclosed herein is a process for making a dicarboxylic acid from a lactone. The process for preparing a dicarboxylic acid according to the present invention generally comprises a step (a) of heating a lactone in the presence of a first catalyst system to produce an alkenoic acid. In a subsequent step (b), the alkenoic acid is contacted with carbon monoxide, water and a second catalyst system to produce a reaction composition comprising the second catalyst system and the dicarboxylic acid.

In embodiments, the present invention includes a process for preparing adipic acid, a Nylon 6-6 monomer, from γ-valerolactone. γ-Valerolactone can be derived from hydrogenation of levulinic acid, a so-called bio-based platform molecule. In turn, levulinic acid can be readily obtained from acid-catalysed decomposition of cellulose or C₆ sugars. Thus, the process according to the present invention provides a method of producing adipic acid from a renewable feedstock.

The term “substantially”, as used herein in relation to an amount, such as in “substantially in the absence” or similar phrases, means that the amount referred to is sufficiently low that it does not significantly affect operation of the invention.

The phrase “substantially in the absence of water”, as used herein, may mean that the concentration of water is sufficiently low that any reduction of this concentration would not increase the yield of alkenoic acid by more than about 10% or more than about 1%. Such concentrations may be less than about 0.1, 0.09, 0.08, 0.07, 0.06, 0.05, 0.04, 0.03, 0.02 or 0.01 moles of water per mole of lactone. This phrase may additionally or alternatively indicate the absence of any added water.

The phrase “substantially in the absence of oxygen”, as used herein, may mean that the concentration of oxygen is less than about 0.1 moles of oxygen per mole of carbon monoxide. Such concentrations may be less than about 0.09, 0.08, 0.07, 0.06, 0.05, 0.04, 0.03, 0.02, 0.01, 0.009, 0.008, 0.007, 0.006, 0.005, 0.003, 0.002, 0.001, 0.0005, 0.0001, 0.00005 and 0.000001 moles of oxygen per mole of carbon monoxide.

The phrase “substantially none of the second catalyst system”, as used herein, may mean that the concentration of the second catalyst system is less than about 1 wt % of the first portion. Such concentrations may be less than about 0.9, 0.8, 0.7, 0.6, 0.5, 0.3, 0.2, 0.1, 0.05, 0.01, 0.005 and 0.0001 wt % of the first portion.

### Step (a): Conversion of Lactone to Alkenoic Acid

In step (a) of the process according to the present invention, a mixture of pentenoic acid isomers can be obtained in high selectivity by heating γ-valerolactone in the presence of an acidic catalyst to drive the equilibrium between the lactone and alkenoic acid toward the formation of the alkenoic acid.

Heating the lactone in step (a) may comprise refluxing, non-reflux heating, distillation or a combination of any two or more of the foregoing. The heating may comprise reactive distillation to produce a distillate comprising the alkenoic acid. The reactive distillation may include refluxing the γ-valerolactone in the presence of an acidic catalyst followed by distillation to produce a distillate comprising the alkenoic acid. The distillation or reactive distillation may represent a purification step.

The lactone used in step (a) may be any suitable lactone. The lactone may have any suitable number of carbon atoms. For example, the lactone may be a propiolactone, a butyrolactone, a valerolactone or a caprolactone. The lactone may have any suitable heterocycle ring size. For example, the lactone may be a β-lactone, a γ-lactone, a δ-lactone or a ε-lactone. For example, the lactone may be β-lactone propiolactone, γ-valerolactone, δ-valerolactone, γ-caprolactone or ε-caprolactone. The process according to the invention may further comprise the step of preparing the lactone prior to step (a).

The alkenoic acid produced in step (a) is determined by the lactone used. For example, if the lactone is γ-valerolactone, the alkenoic acid produced in step (a) is pentenoic acid.

The alkenoic acid produced in step (a) may comprise a single isomer or a plurality of isomers. For example, if the alkenoic acid produced in step (a) comprises pentenoic acid, the alkenoic acid may comprise one or more or 2-pentenoic acid, 3-pentenoic acid and 4-pentenoic acid. The alkenoic acid may comprise 2-pentenoic acid and 3-pentenoic acid, 2-pentenoic acid and 4-pentenoic acid, 3-pentenoic acid and 4-pentenoic acid or 2-pentenoic acid, 3-pentenoic acid and 4-pentenoic acid. Where the alkenoic acid comprises 2-pentenoic acid, the 2-pentenoic acid may comprise one or both of cis-2-pentenoic acid and trans-2-pentenoic acid. Where the alkenoic acid comprises 3-pentenoic acid, the 3-pentenoic acid may comprise one or both of cis-3-pentenoic acid and trans-3-pentenoic acid.

The first catalyst system may be any suitable catalyst system capable of catalysing the conversion of a lactone to an unsaturated carboxylic acid. The first catalyst system may comprise an acidic catalyst. The first catalyst system may be a homogenous catalyst or a heterogeneous solid catalyst. The homogeneous acidic catalyst may comprise one or more of the group consisting of alumina, silica, zeolites (for example, X zeolites, ZSM-5, HZSM-5 and mordenite), clays (for example, montmorillonite), sulphuric acid, p-toluenesulfonic acid or methanesulfonic acid. The first catalyst system may comprise, for example, any of the following mixtures: alumina and silica, alumina and a zeolite, alumina and a clay, alumina and sulphuric acid, alumina and p-toluenesulfonic acid, alumina and methanesulfonic acid, silica and a zeolite, silica and a clay, silica and sulphuric acid, silica and p-toluenesulfonic acid, silica and methanesulfonic acid, a zeolite and a clay, a zeolite and sulphuric acid, a zeolite and p-toluenesulfonic acid, a zeolite and methanesulfonic acid, a clay and sulphuric acid, a clay and p-toluenesulfonic acid, a clay and methanesulfonic acid, sulphuric acid and p-toluenesulfonic acid, sulphuric acid and methanesulfonic acid, p-toluenesulfonic acid and methanesulfonic acid, two or more zeolites, two or more clays, or any combination of the foregoing.

Step (a) may be carried out substantially in the absence of water. In this case, the process according to the present invention may comprise the step of removing water from the lactone and/or from the first catalyst system prior to step (a). The process according to the present invention may comprise the step of removing water from any apparatus used in step (a) prior to this step.

Step (a) may be carried out at any suitable temperature. Step (a) may be carried out at a temperature at or above the normal boiling point of the lactone or step (a) may be carried out at a temperature at or above the boiling point of the lactone at the pressure at which step (a) is carried out. Step (a) may be carried out at a temperature of between about 150° C. and 400° C. For example, step (a) may be carried out at a temperature between about 150° C. and about 160° C., 170° C., 180° C., 190° C., 200° C., 210° C., 220° C., 230° C., 240° C., 250° C., 260° C., 270° C., 280° C., 290° C., 300° C., 310° C., 320° C., 330° C., 340° C., 350° C., 360° C., 370° C., 380° C., 390° C. or 400° C.; between about 160° C. and about 170° C., 180° C., 190° C., 200° C., 210° C., 220° C., 230° C., 240° C., 250° C., 260° C., 270° C., 280° C., 290° C., 300° C., 310° C., 320° C., 330° C., 340° C., 350° C., 360° C., 370° C., 380° C., 390° C. or 400° C.; between about 170° C. and about 180° C., 190° C., 200° C., 210° C., 220° C., 230° C., 240° C., 250° C., 260° C., 270° C., 280° C., 290° C., 300° C., 310° C., 320° C., 330° C., 340° C., 350° C., 360° C., 370° C., 380° C., 390° C. or 400° C.; between about 180° C. and about 190° C., 200° C., 210° C., 220° C., 230° C., 240° C., 250° C., 260° C., 270° C., 280° C., 290° C., 300° C., 310° C., 320° C., 330° C., 340° C., 350° C., 360° C., 370° C., 380° C., 390° C. or 400° C.; between about 190° C. and about 200° C., 210° C., 220° C., 230° C., 240° C., 250° C., 260° C., 270° C., 280° C., 290° C., 300° C., 310° C., 320° C., 330° C., 340° C., 350° C., 360° C., 370° C., 380° C., 390° C. or 400° C.; between about 200° C. and about 210° C., 220° C., 230° C., 240° C., 250° C., 260° C., 270° C., 280° C., 290° C., 300° C., 310° C., 320° C., 330° C., 340° C., 350° C., 360° C., 370° C., 380° C., 390° C. or 400° C.; between about 210° C. and about 220° C., 230° C., 240° C., 250° C., 260° C., 270° C., 280° C., 290° C., 300° C., 310° C., 320° C., 330° C., 340° C., 350° C., 360° C., 370° C., 380° C., 390° C. or 400° C.; between about 210° C. and about 220° C., 230° C., 240° C., 250° C., 260° C., 270° C., 280° C., 290° C., 300° C., 310° C., 320° C., 330° C., 340° C., 350° C., 360° C., 370° C., 380° C., 390° C. or 400° C.; between about 220° C. and about 230° C., 240° C., 250° C., 260° C., 270° C., 280° C., 290° C., 300° C., 310° C., 320° C., 330° C., 340° C., 350° C., 360° C., 370° C., 380° C., 390° C. or 400° C.; between about 230° C. and about 240° C., 250° C., 260° C., 270° C., 280° C., 290° C., 300° C., 310° C., 320° C., 330° C., 340° C., 350° C., 360° C., 370° C., 380° C., 390° C. or 400° C.; between about 240° C. and about 250° C., 260° C., 270° C., 280° C., 290° C., 300° C., 310° C., 320° C., 330° C., 340° C., 350° C., 360° C., 370° C., 380° C., 390° C. or 400° C.; between about 250° C. and about 260° C., 270° C., 280° C., 290° C., 300° C., 310° C., 320° C., 330° C., 340° C., 350° C., 360° C., 370° C., 380° C., 390° C. or 400° C.; between about 260° C. and about 270° C., 280° C., 290° C., 300° C., 310° C., 320° C., 330° C., 340° C., 350° C., 360° C., 370° C., 380° C., 390° C. or 400° C.; between about 270° C. and about 280° C., 290° C., 300° C., 310° C., 320° C., 330° C., 340° C., 350° C., 360° C., 370° C., 380° C., 390° C. or 400° C.; between about 280° C. and about 290° C., 300° C., 310° C., 320° C., 330° C., 340° C., 350° C., 360° C., 370° C., 380° C., 390° C. or 400° C.; between about 290° C. and about 300° C., 310° C., 320° C., 330° C., 340° C., 350° C., 360° C., 370° C., 380° C., 390° C. or 400° C.; between about 300° C. and about 310° C., 320° C., 330° C., 340° C., 350° C., 360° C., 370° C., 380° C., 390° C. or 400° C.; between about 310° C. and about 320° C., 330° C., 340° C., 350° C., 360° C., 370° C., 380° C., 390° C. or 400° C.; between about 320° C. and about 330° C., 340° C., 350° C., 360° C., 370° C., 380° C., 390° C. or 400° C.; between about 330° C. and about 340° C., 350° C., 360° C., 370° C., 380° C., 390° C. or 400° C.; between about 340° C. and about 350° C., 360° C., 370° C., 380° C., 390° C. or 400° C.; between about 350° C. and about 360° C., 370° C., 380° C., 390° C. or 400° C.; between about 360° C. and about 370° C., 380° C., 390° C. or 400° C.; between about 370° C. and about 380° C., 390° C. or 400° C.; between about 380° C. and about 390° C. or 400° C.; or between about 390° C. and about 400° C. Step (a) may be carried out at a temperature of about 150° C., 155° C., 160° C., 165° C., 170° C., 175° C., 180° C., 185° C., 190° C., 195° C., 200° C., 205° C., 210° C., 215° C., 220° C., 225° C., 230° C., 235° C., 240° C., 245° C., 250° C., 255° C., 260° C., 265° C., 270° C., 275° C., 280° C., 285° C., 290° C., 295° C., 300° C., 305° C., 310° C., 315° C., 320° C., 325° C., 330° C., 335° C., 340° C., 345° C., 350° C., 355° C., 360° C., 365° C., 370° C., 375° C., 380° C., 385° C., 390° C., 395° C. or 400° C.

Step (a) may be carried out at any suitable pressure. Step (a) may be carried out at a pressure of between about 0.5 bar and about 30 bar. Step (a) may be carried out at a pressure of between about 0.5 bar and about 1, 2, 3, 4, 5, 6, 7, 8, 9, 10, 15, 20, 25 or 30 bar, between about 1 bar and about 2, 3, 4, 5, 6, 7, 8, 9, 10, 15, 20, 25 or 30 bar, between about 2 bar and about 3, 4, 5, 6, 7, 8, 9, 10, 15, 20, 25 or 30 bar, between about 3 bar and about 4, 5, 6, 7, 8, 9, 10, 15, 20, 25 or 30 bar, between about 4 bar and about 5, 6, 7, 8, 9, 10, 15, 20, 25 or 30 bar, between about 5 bar and about 6, 7, 8, 9, 10, 15, 20, 25 or 30 bar, between about 6 bar and about 7, 8, 9, 10, 15, 20, 25 or 30 bar, between about 7 bar and about 8, 9, 10, 15, 20, 25 or 30 bar, between about 8 bar and about 9, 10, 15, 20, 25 or 30 bar, between about 9 bar and about 10, 15, 20, 25 or 30 bar, between about 10 bar and about 15, 20, 25 or 30 bar, between about 15 bar and about 20, 25 or 30 bar, between about 20 bar and about 25 or 30 bar, or between about 25 bar and about 30 bar. Step (a) may be carried out at a pressure of about 0.5, 0.75, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5 or 30 bar. Step (a) may be carried out at a pressure of about 1 bar.

The first catalyst system and the temperature and pressure of step (a) contribute to determining the percent conversion of lactone to alkenoic acid. The first catalyst system and the temperature and pressure of step (a) may be such that conversion of lactone to alkenoic acid in step (a) is greater than 10%, 20%, 30%, 40%, 50%, 60%, 70%, 75%, 80%, 85%, 90%, 91%, 92%, 93%, 94%, 95%, 96%, 97%, 98%, 99% or 99.5%. The first catalyst system and the temperature and pressure of step (a) may be such that conversion of lactone to alkenoic acid in step (a) is between about 10% and about 20%, 30%, 40%, 50%, 60%, 70%, 75%, 80%, 85%, 90%, 91%, 92%, 93%, 94%, 95%, 96%, 97%, 98%, 99% or 100%, between about 20% and about 30%, 40%, 50%, 60%, 70%, 75%, 80%, 85%, 90%, 91%, 92%, 93%, 94%, 95%, 96%, 97%, 98%, 99% or 100%, between about 30% and about 40%, 50%, 60%, 70%, 75%, 80%, 85%, 90%, 91%, 92%, 93%, 94%, 95%, 96%, 97%, 98%, 99% or 100%, between about 40% and about 50%, 60%, 70%, 75%, 80%, 85%, 90%, 91%, 92%, 93%, 94%, 95%, 96%, 97%, 98%, 99% or 100%, between about 50% and about 60%, 70%, 75%, 80%, 85%, 90%, 91%, 92%, 93%, 94%, 95%, 96%, 97%, 98%, 99% or 100%, between about 60% and about 70%, 75%, 80%, 85%, 90%, 91%, 92%, 93%, 94%, 95%, 96%, 97%, 98%, 99% or 100%, between about 70% and about 75%, 80%, 85%, 90%, 91%, 92%, 93%, 94%, 95%, 96%, 97%, 98%, 99% or 100%, between about 75% and about 80%, 85%, 90%, 91%, 92%, 93%, 94%, 95%, 96%, 97%, 98%, 99% or 100%, between about 80% and about 85%, 90%, 91%, 92%, 93%, 94%, 95%, 96%, 97%, 98%, 99% or 100%, between about 85% and about 90%, 91%, 92%, 93%, 94%, 95%, 96%, 97%, 98%, 99% or 100%, between about 90% and about 91%, 92%, 93%, 94%, 95%, 96%, 97%, 98%, 99% or 100%, between about 91% and about 92%, 93%, 94%, 95%, 96%, 97%, 98%, 99% or 100%, between about 92% and about 93%, 94%, 95%, 96%, 97%, 98%, 99% or 100%, between about 93% and about 94%, 95%, 96%, 97%, 98%, 99% or 100%, between about 94% and about 95%, 96%, 97%, 98%, 99% or 100%, between about 95% and about 96%, 97%, 98%, 99% or 100%, between about 96% and about 97%, 98%, 99% or 100%, between about 97% and about 98%, 99% or 100%, between about 98% and about 99% or 100%, or between about 99% and about 100%. The first catalyst system and the temperature and pressure of step (a) may be such that conversion of lactone to alkenoic acid in step (a) is about 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or 100%.

Where the conversion of lactone to alkenoic acid in step (a) is less than 100%, step (a) may produce a reaction composition of the alkenoic acid and unreacted lactone. In this case, the process according to the invention may further comprise a step (a1), subsequent to step (a), of separating part or substantially all the unreacted lactone from the pentenoic acid produced in step (a). The separation of the unreacted lactone in step (a1) may be achieved by any suitable means. For example, the separation in step (a1) may be achieved using a flashing unit, a normal distillation unit, a vacuum distillation unit, chromatography or crystallisation.

Where the process of the present invention comprises step (a1), the process may comprise a further step (a2), subsequent to step (a1), of recycling the separated unreacted lactone to step (a).

### Step (b): Conversion of Alkenoic Acid to Diacid

In step (b) of the process according to the present invention the dicarboxylic acid produced is determined by the alkenoic acid used and, therefore, the lactone used in step (a). For example, if the lactone used in step (a) is γ-valerolactone, the alkenoic acid produced in step (a) is pentenoic acid and the dicarboxylic acid produced in step (b) is adipic acid.

Step (b) may include preparation of the second catalyst system in situ. In this case, step (b) may include combining the catalyst precursors. The process according to the invention may comprise an additional step of preparing a palladium catalyst. In this case, the additional step may include combining the catalyst precursors.

The second catalyst system may be any suitable catalyst system. The second catalyst system may comprise a catalyst based on an element from group 9 or group 10 of the periodic table. For example, the second catalyst system may comprise a palladium catalyst, a platinum catalyst, a nickel catalyst, an iridium catalyst or a rhodium catalyst. The second catalyst system may comprise a palladium catalyst. The second catalyst system may comprise a palladium catalyst of formula (I):

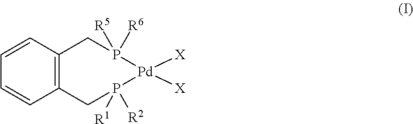

wherein X is a ligand and each of R¹, R², R⁵ and R⁶ may independently be an optionally substituted organic group or R¹ and R² and/or R⁵ and R⁶, together with the P atom to which they are attached, may form a cyclic group.

In formula (I), R¹, R², R⁵ and R⁶ may each independently represent a tertiary alkyl, cycloalkyl, heterocyclyl, aryl or heteroaryl. R¹, R², R⁵ and R⁶ may each independently represent tertiary butyl, adamantyl or phenyl, or, together with the P atom to which they are attached, form a phospha trioxa-adamantane group of formula (III):

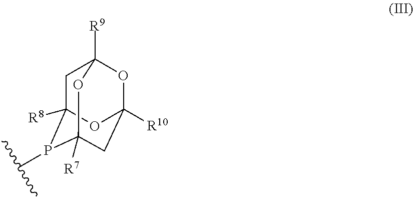

wherein R⁷, R⁸, R⁹ and R¹⁰ may each independently represent an optionally substituted hydrocarbyl group.

In formula (III), R⁷, R⁸, R⁹ and R¹⁰ may, for example, each independently represent an optionally substituted alkyl, haloalkyl, aryl, heteroaryl, cycloalkyl or heterocyclyl. For example, R⁷, R⁸, R⁹ and R¹⁰ may each independently represent an optionally substituted C₁-C₆-alkyl, C₁-C₆-haloalkyl, C₆-C₁₀-aryl, C₄-C₈-heteroaryl, C₄-C₁₀-cycloalkyl or C₄-C₈-heterocyclyl. The haloalkyl may comprise one or more halogen atoms selected from the group consisting of F, Cl and Br. For example, R⁷, R⁸, R⁹ and R¹⁰ may each independently represent CF₃. The heteroaryl and heterocyclyl may comprise N, S or O atoms.

The process according to the invention may comprise the step of preparing a palladium catalyst of formula (I) by combining a palladium compound, a bidentate diphosphine and an acid. This may be conducted in situ or may be conducted separately. Step (b) may include preparing a palladium catalyst of formula (I) by combining a palladium compound, a bidentate diphosphine and an acid.

X in formula (I) may be derived from the acid used to prepare the palladium catalyst.

The bidentate diphosphine used to prepare the palladium catalyst may be any suitable bidentate diphosphine. The bidentate diphosphine may have the formula (II):

(R¹)(R²)P—R³—Ar—R⁴—P(R⁵)(R⁶) (II)

wherein
Ar is an optionally substituted aromatic group;
R¹ and R² may either be the same or different and may represent a tertiary alkyl or, together with the P atom to which they are attached, may form a phospha trioxa-adamantane group having formula (III):

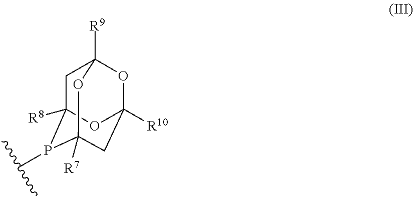

wherein R⁷, R⁸, R⁹ and R¹⁰ may each independently represent an optionally substituted hydrocarbyl group;
R³ and R⁴ each independently represent an optionally substituted alkylene group; and
R⁵ and R⁶ each independently represent an optionally substituted organic group or together with the P atom to which they are attached, form a cyclic group.

In formula (II), R³ and R⁴ may occupy ortho positions on Ar.

In formula (II), R⁵ and R⁶ may each independently represent a tertiary alkyl, cycloalkyl, heterocyclyl or aryl. R⁵ and R⁶ may each independently represent tertiary butyl, adamantyl or phenyl, or, together with the P atom to which they are attached, form a phospha trioxa-adamantane group of formula (III). The bidentate diphosphine may, for example, be 1,2-bis[di(t-butyl)phosphinomethyl]benzene.

The palladium compound used to prepare the palladium catalyst may be any suitable palladium compound. The palladium compound may be selected from the group consisting of palladium (ii) compounds (e.g., palladium carboxylates) and palladium(0) compounds. The palladium compound may be palladium acetate, palladium tosylate, tris(dibenzylideneacetone)dipalladium(0) or palladium acetylacetonate.

The second catalyst system may comprise a palladium catalyst derived from palladium acetate, 1,2-bis[di(t-butyl)phosphinomethyl]benzene, and methane sulfonic acid (Scheme 1).

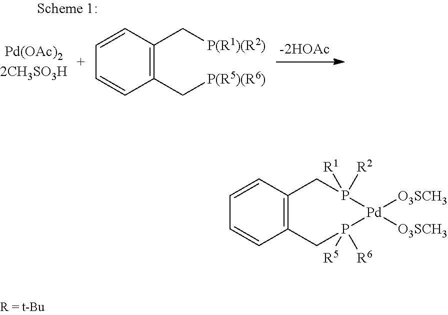

The palladium catalyst may be prepared in situ by combining palladium acetate, 1,2-bis[di(t-butyl)phosphinomethyl]benzene, and methane sulfonic acid. The process according to the invention may comprise an additional step of preparing the palladium catalyst by combining palladium acetate, 1,2-bis[di(t-butyl)phosphinomethyl]benzene, and methane sulfonic acid.

The acid used to prepare the palladium catalyst may be any suitable acid, having any suitable pKa. The acid may be a monoprotic acid or a polyprotic acid. The acid may have a pKa (measured in water at 18° C.) of less than about 5, 4, 3, 2, 1, 0, −1, −2, −5, −10 or −15. The acid may have a pKa (measured in water at 18° C.) of between about 5 and about 4, 3, 2, 1, 0, −1, −2, −5, −10 or −15, between about 4 and about 3, 2, 1, 0, −1, −2, −5, −10 or −15, between about 3 and about 2, 1, 0, −1, −2, −5, −10 or −15, between about 2 and about 1, 0, −1, −2, −5, −10 or −15, between about 1 and about 0, −1, −2, −5, −10 or −15, between about 0 and about −1, −2, −5, −10 or −15, between about −1 and −2, −5, −10 or −15, between about −2 and about −5, −10 or −15, between about −5 and about −10 or −15, or between about −10 and −15. The acid may have a pKa (measured in water at 18° C.) of about 5, 4, 3, 2, 1, 0, −1, −2, −5, −10 or −15. The acid may be selected from the group consisting of sulfonic acids, sulphuric acids, phosphorous acids and carboxylic acids. The acid may be a C₁-C₁₀ aliphatic acid. The acid may be selected from the group consisting of methanesulfonic acid, triflic acid, trifluoroacetic acid and acetic acid. The acid may be the alkenoic acid produced in step (a) of the process of the present invention.

The acid used to prepare the palladium catalyst may be present at any suitable concentration relative to the palladium compound and bidentate diphosphine. The acid may be present in molar excess relative to the palladium compound (i.e., the molar ratio of the acid to the palladium compound is greater than 1:1). The molar ratio of the acid to the palladium compound may be greater than about 1 (i.e., 1:1), 1.5, 2, 3, 4, 5, 6, 7, 8, 9, 10, 20, 30, 40, 50, 60, 70, 80, 90, 100, 200, 300, 400, 500, 750, 1000, 5000 or 10000. The molar ratio of the acid to the palladium compound may be between about 1 and about 1.5, 2, 3, 4, 5, 6, 7, 8, 9, 10, 20, 30, 40, 50, 60, 70, 80, 90, 100, 200, 300, 400, 500, 750, 1000, 5000 or 10000, between about 1.5 and about 2, 3, 4, 5, 6, 7, 8, 9, 10, 20, 30, 40, 50, 60, 70, 80, 90, 100, 200, 300, 400, 500, 750, 1000, 5000 or 10000, between about 2 and about 3, 4, 5, 6, 7, 8, 9, 10, 20, 30, 40, 50, 60, 70, 80, 90, 100, 200, 300, 400, 500, 750, 1000, 5000 or 10000, between about 3 and about 4, 5, 6, 7, 8, 9, 10, 20, 30, 40, 50, 60, 70, 80, 90, 100, 200, 300, 400, 500, 750, 1000, 5000 or 10000, between about 4 and about 5, 6, 7, 8, 9, 10, 20, 30, 40, 50, 60, 70, 80, 90, 100, 200, 300, 400, 500, 750, 1000, 5000 or 10000, between about 5 and about 6, 7, 8, 9, 10, 20, 30, 40, 50, 60, 70, 80, 90, 100, 200, 300, 400, 500, 750, 1000, 5000 or 10000, between about 6 and about 7, 8, 9, 10, 20, 30, 40, 50, 60, 70, 80, 90, 100, 200, 300, 400, 500, 750, 1000, 5000 or 10000, between about 7 and about 8, 9, 10, 20, 30, 40, 50, 60, 70, 80, 90, 100, 200, 300, 400, 500, 750, 1000, 5000 or 10000, between about 8 and about 9, 10, 20, 30, 40, 50, 60, 70, 80, 90, 100, 200, 300, 400, 500, 750, 1000, 5000 or 10000, between about 9 and about 10, 20, 30, 40, 50, 60, 70, 80, 90, 100, 200, 300, 400, 500, 750, 1000, 5000 or 10000, between about 10 and about 20, 30, 40, 50, 60, 70, 80, 90, 100, 200, 300, 400, 500, 750, 1000, 5000 or 10000, between about 20 and about 30, 40, 50, 60, 70, 80, 90, 100, 200, 300, 400, 500, 750, 1000, 5000 or 10000, between about 30 and about 40, 50, 60, 70, 80, 90, 100, 200, 300, 400, 500, 750, 1000, 5000 or 10000, between about 40 and about 50, 60, 70, 80, 90, 100, 200, 300, 400, 500, 750, 1000, 5000 or 10000, between about 50 and about 60, 70, 80, 90, 100, 200, 300, 400, 500, 750, 1000, 5000 or 10000, between about 60 and about 70, 80, 90, 100, 200, 300, 400, 500, 750, 1000, 5000 or 10000, between about 70 and about 80, 90, 100, 200, 300, 400, 500, 750, 1000, 5000 or 10000, between about 80 and about 90, 100, 200, 300, 400, 500, 750, 1000, 5000 or 10000, between about 90 and about 100, 200, 300, 400, 500, 750, 1000, 5000 or 10000, between about 100 and about 200, 300, 400, 500, 750, 1000, 5000 or 10000, between about 200 and about, 300, 400, 500, 750, 1000, 5000 or 10000, between about 300 and about 400, 500, 750, 1000, 5000 or 10000, between about 400 and about 500, 750, 1000, 5000 or 10000, between about 500 and about 750, 1000, 5000 or 10000, between about 750 and about 1000, 5000 or 10000, between about 1000 and about 5000 or 10000, or between about 5000 and about 10000. The molar ratio of the acid to the palladium compound may be about 1, 1.5, 2, 3, 4, 5, 6, 7, 8, 9, 10, 20, 30, 40, 50, 60, 70, 80, 90, 100, 200, 300, 400, 500, 750, 1000, 5000 or 10000.

Step (b) may be carried out substantially in the absence of oxygen. In this case, the process according to the present invention may comprise the step of removing oxygen from the water and second catalyst system prior to step (b). The process according to the present invention may comprise the step of removing oxygen from any apparatus used in step (b) prior to this step. The step of removing oxygen may comprise any suitable means of removing oxygen. For example, the step may comprise purging with an inert gas, one or more freeze-pump-thaw cycles or use of an oxygen scavenger. Step (b) may be carried out in an inert atmosphere. For example, step (b) may be carried out in an argon atmosphere.

Step (b) may be carried out at any suitable temperature. Step (b) may be carried out at a temperature of between about 30° C. and about 150° C. For example, step (b) may be carried out at a temperature of between about 30° C. and about 40° C., 50° C., 60° C., 70° C., 80° C., 90° C., 100° C., 110° C., 120° C., 130° C., 140° C. or 150° C., between about 40° C. and about 50° C., 60° C., 70° C., 80° C., 90° C., 100° C., 110° C., 120° C., 130° C., 140° C. or 150° C., between about 50° C. and about 60° C., 70° C., 80° C., 90° C., 100° C., 110° C., 120° C., 130° C., 140° C. or 150° C., between about 60° C. and about 70° C., 80° C., 90° C., 100° C., 110° C., 120° C., 130° C., 140° C. or 150° C., between about 70° C. and about 80° C., 90° C., 100° C., 110° C., 120° C., 130° C., 140° C. or 150° C., between about 80° C. and about 90° C., 100° C., 110° C., 120° C., 130° C., 140° C. or 150° C., between about 90° C. and about 100° C., 110° C., 120° C., 130° C., 140° C. or 150° C., between about 100° C. and about 110° C., 120° C., 130° C., 140° C. or 150° C., between about 110° C. and about 120° C., 130° C., 140° C. or 150° C., between about 120° C. and about 130° C., 140° C. or 150° C., between about 130° C. and about 140° C. or 150° C., or between about 140° C. and about 150° C. Step (b) may be carried out at a temperature of about 30° C., 35° C., 40° C., 45° C., 50° C., 55° C., 60° C., 65° C., 70° C., 75° C., 80° C., 85° C., 90° C., 95° C., 100° C., 105° C., 110° C., 115° C., 120° C., 125° C., 130° C., 135° C., 140° C., 145° C. or 150° C.

Step (b) may be carried out at any suitable pressure. Step (b) may be carried out at a pressure of between about 1 bar and about 150 bar. For example, step (b) may be carried out at a pressure of between about 1 bar and about 2, 3, 5, 10, 20, 30, 40, 50, 60, 70, 80, 90, 100, 110, 120, 130, 140 or 150 bar, between about 2 bar and about 3, 5, 10, 20, 30, 40, 50, 60, 70, 80, 90, 100, 110, 120, 130, 140 or 150 bar, between about 3 bar and about 5, 10, 20, 30, 40, 50, 60, 70, 80, 90, 100, 110, 120, 130, 140 or 150 bar, between about 5 bar and about 10, 20, 30, 40, 50, 60, 70, 80, 90, 100, 110, 120, 130, 140 or 150 bar, between about 10 bar and about 20, 30, 40, 50, 60, 70, 80, 90, 100, 110, 120, 130, 140 or 150 bar, between about 20 bar and about 30, 40, 50, 60, 70, 80, 90, 100, 110, 120, 130, 140 or 150 bar, between about 30 bar and about 40, 50, 60, 70, 80, 90, 100, 110, 120, 130, 140 or 150 bar, between about 40 bar and about 50, 60, 70, 80, 90, 100, 110, 120, 130, 140 or 150 bar, between about 50 bar and about 60, 70, 80, 90, 100, 110, 120, 130, 140 or 150 bar, between about 60 bar and about 70, 80, 90, 100, 110, 120, 130, 140 or 150 bar, between about 80 bar and about 90, 100, 110, 120, 130, 140 or 150 bar, between about 90 bar and about 100, 110, 120, 130, 140 or 150 bar, between about 100 bar and about 110, 120, 130, 140 or 150 bar, between about 110 bar and about 120, 130, 140 or 150 bar, between about 120 bar and about 130, 140 or 150 bar, between about 130 bar and about 140 or 150 bar, or between about 140 bar and about 150 bar. Step (b) may be carried out a temperature of about 1, 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145 or 150 bar.

The second catalyst system and the temperature and pressure of step (b) contribute to determining the percent conversion of alkenoic acid to dicarboxylic acid. The second catalyst system, the temperature of step (b) and the pressure of step (b) may be such that the conversion of alkenoic acid to dicarboxylic acid in step (b) is greater than 10%, 20%, 30%, 40%, 50%, 60%, 70%, 75%, 80%, 85%, 90%, 91%, 92%, 93%, 94%, 95%, 96%, 97%, 98%, 99% or 99.5%. The second catalyst system, the temperature of step (b) and the pressure of step (b) may be such that the conversion of alkenoic acid to dicarboxylic acid in step (b) is between about 10% and about 20%, 30%, 40%, 50%, 60%, 70%, 75%, 80%, 85%, 90%, 91%, 92%, 93%, 94%, 95%, 96%, 97%, 98%, 99% or 100%, between about 20% and about 30%, 40%, 50%, 60%, 70%, 75%, 80%, 85%, 90%, 91%, 92%, 93%, 94%, 95%, 96%, 97%, 98%, 99% or 100%, between about 30% and about 40%, 50%, 60%, 70%, 75%, 80%, 85%, 90%, 91%, 92%, 93%, 94%, 95%, 96%, 97%, 98%, 99% or 100%, between about 40% and about 50%, 60%, 70%, 75%, 80%, 85%, 90%, 91%, 92%, 93%, 94%, 95%, 96%, 97%, 98%, 99% or 100%, between about 50% and about 60%, 70%, 75%, 80%, 85%, 90%, 91%, 92%, 93%, 94%, 95%, 96%, 97%, 98%, 99% or 100%, between about 60% and about 70%, 75%, 80%, 85%, 90%, 91%, 92%, 93%, 94%, 95%, 96%, 97%, 98%, 99% or 100%, between about 70% and about 75%, 80%, 85%, 90%, 91%, 92%, 93%, 94%, 95%, 96%, 97%, 98%, 99% or 100%, between about 75% and about 80%, 85%, 90%, 91%, 92%, 93%, 94%, 95%, 96%, 97%, 98%, 99% or 100%, between about 80% and about 85%, 90%, 91%, 92%, 93%, 94%, 95%, 96%, 97%, 98%, 99% or 100%, between about 85% and about 90%, 91%, 92%, 93%, 94%, 95%, 96%, 97%, 98%, 99% or 100%, between about 90% and about 91%, 92%, 93%, 94%, 95%, 96%, 97%, 98%, 99% or 100%, between about 91% and about 92%, 93%, 94%, 95%, 96%, 97%, 98%, 99% or 100%, between about 92% and about 93%, 94%, 95%, 96%, 97%, 98%, 99% or 100%, between about 93% and about 94%, 95%, 96%, 97%, 98%, 99% or 100%, between about 94% and about 95%, 96%, 97%, 98%, 99% or 100%, between about 95% and about 96%, 97%, 98%, 99% or 100%, between about 96% and about 97%, 98%, 99% or 100%, between about 97% and about 98%, 99% or 100%, between about 98% and about 99% or 100%, or between about 99% and about 100%. The second catalyst system, the temperature of step (b) and the pressure of step (b) may be such that the conversion of alkenoic acid to dicarboxylic acid in step (b) is about 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or 100%.

Where step (a) produces a reaction composition of the alkenoic acid and unreacted lactone, the reaction composition produced in step (b) may comprise part or substantially all of the unreacted lactone from step (a). In this case, the process of the present invention may further comprise a step (b1), subsequent to step (b), of separating part or substantially all the unreacted lactone from one the dicarboxylic acid present in the reaction composition. Step (b1) may also comprise separating part or substantially all the unreacted lactone from the second catalyst system and/or any other components present in the reaction composition, such as unreacted alkenoic acid. The separation of the unreacted lactone in step (b1) may be achieved by any suitable means. For example, the separation in step (b1) may be achieved using a flashing unit, normal distillation unit or vacuum distillation unit.

Where the process of the present invention comprises step (b1), the process may comprise a further step (b2), subsequent to step (b1), of recycling the separate unreacted lactone to step (a) and/or step (b).

Where the conversion of alkenoic acid to dicarboxylic acid in step (b) is less than 100%, step (b) may produce a reaction composition comprising unreacted alkenoic acid. In this case, the process of the present invention may further comprise a step (b3), subsequent to step (b), of separating part or substantially all the unreacted alkenoic acid from the dicarboxylic acid. Step (b3) may also comprise separating part or substantially all the unreacted alkenoic acid from the second catalyst system and/or any other components present in the reaction composition, such as unreacted lactone. The separation of the unreacted alkenoic acid in step (b3) may be achieved by any suitable means. For example, the separation in step (b3) may be achieved using a flashing unit, normal distillation unit or vacuum distillation unit.

Where the process of the present invention comprises step (b3), the process may comprise a further step (b4), subsequent to step (b1), of recycling the separated unreacted alkenoic acid to step (a) and/or step (b).

The process according to the present invention may further comprise the step (c), subsequent to step (b), of separating the dicarboxylic acid from the remainder of the reaction composition to produce a first portion comprising the dicarboxylic acid and a second portion comprising the second catalyst system. The first portion, comprising the dicarboxylic acid, may comprise substantially none of the second catalyst system. The process according to the invention may further comprise the step of washing the first portion.

Step (b) may be carried out in a solvent. The solvent may be such that the dicarboxylic acid separates from the unreacted alkenoic acid in step (c) by crystallisation. The solvent may be such that the dicarboxylic acid can be separated from the unreacted alkenoic acid in step (c) by reducing the temperature of the reaction composition, thereby causing the dicarboxylic acid to crystallise. The solvent may, for example, be unreacted lactone from step (a), additional lactone of the type used in step (a) or some other solvent in which the dicarboxylic acid is less soluble than the alkenoic acid under certain conditions, for example bis(2-methoxyethyl) ether (diglyme). Step (c) may comprise reducing the temperature of the reaction composition, by any suitable means, such that the dicarboxylic acid crystallises.

Where the process of the present invention comprises step (c), the process may further comprise the step (d) of recycling the second portion, comprising the second catalyst system, to step (b).

### Process for Preparing Adipic Acid

The process according to the present invention may be used for preparing adipic acid. When used for preparing adipic acid, the process according to the invention may comprise the steps of: (a) heating γ-valerolactone in the presence of an acidic catalyst to produce a mixture of pentenoic acid isomers and (b) carbonylation of the mixture of pentenoic acid isomers to adipic acid in the presence of a palladium catalyst and water to generate adipic acid with high selectivity (Scheme 2).

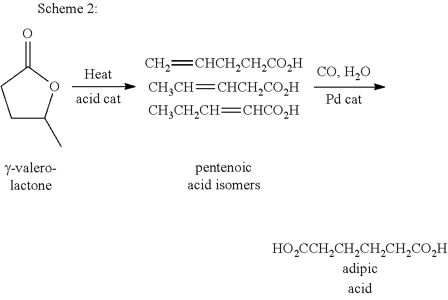

Without wishing to be bound by theory, the high selectivity to adipic acid may be attributed to rapid equilibration between pentenoic acid isomers and the slow carbonylation of internal olefinic carbons relative to the terminal olefinic carbon, i.e. k₆>>k₁, k₂, k₃, k₄, and k₅ (Scheme 3).

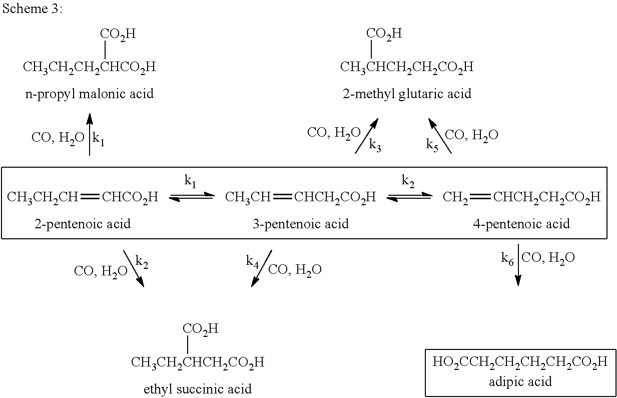

When used for preparing adipic acid, the process according to the invention may further comprise the step of preparing the γ-valerolactone by hydrogenation of levulinic acid. The process according to the invention may further comprise the step of preparing the levulinic acid by acid catalysed hydrolysis of cellulose. The process according to the invention may further comprise the step of preparing the cellulose by cracking lignocellulose (Scheme 4).

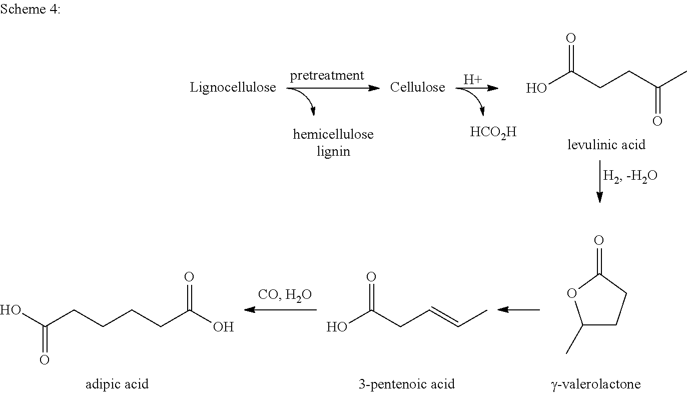

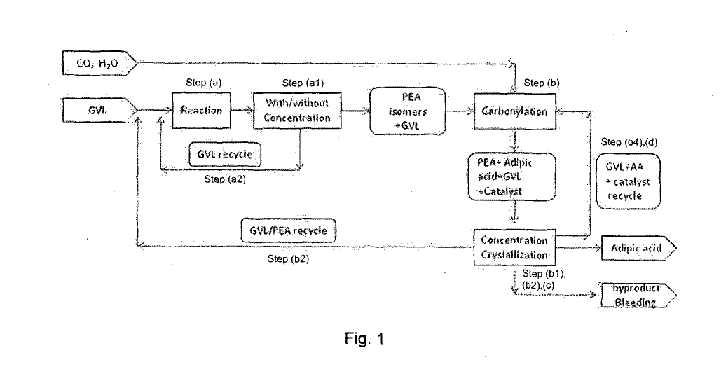

When used for preparing adipic acid, a suitable process according to the invention may comprise the following steps (FIG. 1):
(a) heating γ-valerolactone in the presence of a first catalyst system to produce a mixture of pentenoic acid isomers and, optionally, unreacted γ-valerolactone;
(a1) optionally separating part or substantially all of any unreacted γ-valerolactone from the pentenoic acid;
(a2) optionally recycling the separated unreacted γ-valerolactone from step (a1) to step (a);
(b) contacting the pentenoic acid with carbon monoxide, water and a second catalyst system to produce a reaction composition comprising the second catalyst system, adipic acid and, optionally, unreacted γ-valerolactone and/or unreacted pentenoic acid and/or unreacted carbon monoxide and/or unreacted water;
(b1) optionally separating part or substantially all of the unreacted γ-valerolactone from the adipic acid;
(b2) optionally recycling the separated unreacted γ-valerolactone to step (a) and/or step (b);
(b3) optionally separating part or substantially all the unreacted pentenoic acid from the adipic acid;
(b4) optionally recycling the separated unreacted pentenoic acid to step (a) and/or step (b);
(c) optionally separating the adipic acid from the remainder of the reaction composition to produce a first portion comprising the adipic acid and a second portion comprising the second catalyst system; and
(d) optionally recycling the second portion, comprising the second catalyst system, to step (b).

The process according to the invention illustrated in FIG. 1 may further comprise the step of washing the first portion obtained in step (c).

The adipic acid produced by the process according to the invention may be used as a monomer in a process for the preparation of a polymer. For example, the adipic acid may be used in the preparation of Nylon 6-6, having the formula:

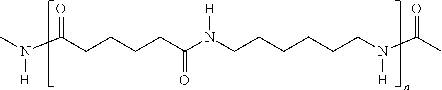

The adipic acid may be used in a process comprising copolymerising the adipic acid with hexamethylenediamine to form Nylon 6-6.

### EXAMPLES

### Example 1

### Preparation of Pentenoic Acid Isomers Via Catalytic Distillation of γ-Valerolactone

A mixture of 200 ml γ-valerolactone and 10 g of silica-alumina (grade 135) was allowed to reflux for 120 min in a distillation apparatus equipped with a fractionating column (24 mm I.D.) packed with 1200 mm of stainless steel PRO-Pak® packing from ACE Glass. The mixture was then distilled at a bottom temperature of around 210° C. to yield 20 ml of distillate over a period of 200 min. Gas chromatography (GC) analysis showed that the distillate contained 52.6% γ-valerolactone, 21.1% 2-pentenoic acid, 16.0% 3-pentenoic acid, 9.9% 4-pentenoic acid and 0.3% other impurities.

### Example 2

### Preparation of Pentenoic Acid Isomers Via Catalytic Distillation of γ-Valerolactone

A mixture of 400 ml γ-valerolactone and 40 g of silica-alumina (grade 135) was allowed to reflux for 120 min in a distillation apparatus equipped with a fractionating column (24 mm I.D.) packed with 1200 mm of stainless steel PRO-Pak® packing from ACE Glass. The mixture was then distilled at a bottom temperature of around 210° C. to yield 35 ml of distillate over a period of 240 min. GC-analysis showed that the distillate contained 3.3% γ-valerolactone, 24.3% 2-pentenoic acid, 40.5% 3-pentenoic acid, 31.1% 4-pentenoic acid and 0.8% other impurities.

### Example 3

### Preparation of Pentenoic Acid Isomers Via Catalytic Distillation of γ-Valerolactone

The method of Example 2 was repeated and 45 ml of distillate was collected. GC-analysis showed that the distillate contained 14.8% γ-valerolactone, 32.2% 2-pentenoic acid, 35.4% 3-pentenoic acid, 17.1% 4-pentenoic acid and 0.5% other impurities.

### Example 4

### Preparation of Pentenoic Acid Isomers Via Catalytic Distillation of γ-Valerolactone

A mixture of 400 ml γ-valerolactone and 40 g of an aluminosilicate zeolite catalyst (ZSM-5) was allowed to reflux for 120 min in a distillation apparatus equipped with a fractionating column (24 mm I.D.) packed with 1200 mm of stainless steel PRO-Pak® packing from ACE Glass. The mixture was then distilled at a bottom temperature of around 210° C. to yield 35 ml of distillate over a period of 240 min. GC-analysis showed that the distillate contained 23.8% γ-valerolactone, 27.6% 2-pentenoic acid, 32.6% 3-pentenoic acid, 15.3% 4-pentenoic acid and 0.7% other impurities.

### Example 5

### Carbonylation of an Isomeric Mixture of Pentenoic Acids to Adipic Acid

A stainless steel 300 ml Parr reactor was charged with degassed diglyme (40 ml), degassed deionised water (5.0 ml, 228 mmol), and degassed distillate prepared by the method described in Example 1 (13.6 ml, 61.6 mmol pentenoic acid isomers: distillate composition by GC-analysis: 12% 2-pentenoic acid, 20% 3-pentenoic acid, 14% 4-pentenoic acid and 54% γ-valerolactone) under a stream of argon gas. The Parr reactor was evacuated and refilled with CO (2 bar). A yellow solution of catalyst (0.2 mol % Pd relative to total pentenoic acid isomers) consisting of palladium acetate (30.5 mg, 0.14 mmol), 1,2-bis[di(t-butyl)phosphinomethyl]benzene (108.2 mg, 0.27 mmol), and methane sulfonic acid (0.1 mL, 1.5 mmol) in diglyme (10 ml) was injected into the reactor under a stream of CO gas. The Parr reactor was then pressurised with CO (60 bar). The reaction mixture was stirred at 1000 rpm. The Parr reactor was heated at 105° C. for 5 h. After 5 h, the reactor was cooled, vented and opened to air. A yellow reaction mixture was obtained, which was placed in a refrigerator to allow the adipic acid formed to crystallise. The crude adipic acid was filtered, washed with ethyl acetate and dried under vacuum at 60° C. to give 1.999 g of adipic acid crystals (13.7 mmol, m.p. 151.4° C.-154.8° C.). Analysis of the crude reaction mixture by ¹³C NMR and GC showed adipic acid as the only product, with unreacted γ-valerolactone from the distillate also present.

### Example 6

### Carbonylation of an Isomeric Mixture of Pentenoic Acids to Adipic Acid

A stainless steel 300 ml Parr reactor was charged with degassed diglyme (40 ml), degassed deionised water (5.0 ml, 228 mmol), and degassed distillate prepared by the method described in Example 1 (15.0 ml, 84.2 mmol pentenoic acid isomers: distillate composition by ¹H NMR: 9% 2-pentenoic acid, 29% 3-pentenoic acid, 19% 4-pentenoic acid and 43%; γ-valerolactone) under a stream of argon gas. A yellow solution of catalyst (0.2 mol % Pd relative to total pentenoic acid isomers) consisting of palladium acetate (39.9 mg, 0.18 mmol), 1,2-bis[di(t-butyl)phosphinomethyl]benzene (133.0 mg, 0.34 mmol), and methane sulfonic acid (0.1 ml, 1.5 mmol) in diglyme (14 ml) was then injected into the reactor under a stream of argon gas. The Parr reactor was then pressurised with CO (60 bar). The reaction mixture was stirred at 1000 rpm. The Parr reactor was heated at 105° C. for 5 h. After 5 h, the reactor was cooled, vented and opened to air. A yellow reaction mixture was obtained, from which white adipic acid crystals were separated and washed with acetonitrile. The yellow mother liquor was placed in a refrigerator to further crystallise out the remaining adipic acid. The crude adipic acid was filtered and washed with acetonitrile. The combined adipic acid fractions were dried under vacuum at 60° C. to give 5.855 g of adipic acid crystals (40.1 mmol, m.p. 151.5° C.-155.8° C.). Analysis of the crude reaction mixture by ¹³C NMR showed adipic acid as the only product, with unreacted γ-valerolactone from the distillate also present.

### Example 7

### Carbonylation of 2-Pentenoic Acid to Adipic Acid

The method of Example 6 was repeated using degassed 2-pentenoic acid (15.0 ml, 148 mmol) instead of the mixture of pentenoic acid isomers. The yellow solution of catalyst (0.2 mol % Pd relative to 2-pentenoic acid) consisting of palladium acetate (66.7 mg, 0.30 mmol), 1,2-bis[di(t-butyl)phosphinomethyl]benzene (234.8 mg, 0.60 mmol), and methane sulfonic acid (0.1 mL, 1.5 mmol) in diglyme (18 ml) was injected into the reactor under a stream of argon gas. After 5 h, the reactor was cooled, vented and opened to air. A yellow reaction mixture was obtained, from which white adipic acid crystals were separated and washed with acetonitrile. The yellow mother liquor was placed in a refrigerator to further crystallise out the remaining adipic acid. The crude adipic acid was filtered and washed with acetonitrile. The combined adipic acid fractions were dried under vacuum at 60° C. to give 12.16 g of adipic acid crystals (85.5 mmol). Analysis of the crude reaction mixture by ¹³C NMR showed the presence of unreacted 2-pentenoic acid as well as adipic acid.

### Example 8

### Carbonylation of 3-Pentenoic Acid to Adipic Acid

The method of Example 6 was repeated using degassed 3-pentenoic acid (15.0 ml, 148 mmol) instead of the mixture of pentenoic acid isomers. The yellow solution of catalyst (0.2 mol % Pd relative to 3-pentenoic acid) consisting of palladium acetate (58.6 mg, 0.26 mmol), 1,2-bis[di(t-butyl)phosphinomethyl]benzene (198.9 mg, 0.50 mmol), and methane sulfonic acid (0.1 mL, 1.5 mmol) in diglyme (18 ml) was injected into the reactor under a stream of CO gas. After 5 h, the reactor was cooled, vented and opened to air. White adipic acid crystals in a yellow reaction mixture were obtained. The crude adipic acid was filtered, washed with methanol and dried under vacuum at 60° C. to give 9.452 g of adipic acid crystals (64.7 mmol). Analysis of the crude reaction mixture by GC showed the presence of 2-pentenoic acid in addition to the adipic acid.

### Example 9

### Carbonylation of 4-Pentenoic Acid to Adipic Acid

The method of Example 6 was repeated using degassed 4-pentenoic acid (14.0 ml, 137 mmol) instead of the mixture of pentenoic acid isomers. The yellow solution of catalyst (0.2 mol % Pd relative to 4-pentenoic acid) consisting of palladium acetate (62.1 mg, 0.28 mmol), 1,2-bis[di(t-butyl)phosphinomethyl]benzene (216.9 mg, 0.55 mmol), and methane sulfonic acid (0.1 mL, 1.5 mmol) in diglyme (15 ml) was injected into the reactor under a stream of argon gas. After 5 h, the reactor was cooled, vented and opened to air. White adipic acid crystals in a yellow reaction mixture were obtained. The white adipic acid crystals were separated from the yellow reaction mixture and washed with acetonitrile. The yellow mother liquor was placed in a refrigerator to further crystallise out the adipic acid. Crude adipic acid was filtered and washed with acetonitrile. The combined adipic acid fractions were dried under vacuum at 60° C. to give 14.093 g of adipic acid crystals (96.4 mmol). Analysis of the crude reaction mixture by ¹³C NMR showed the presence of adipic acid and some 2-methylglutaric acid by-product.

### Example 10

### Carbonylation of 3-Pentenoic Acid to Adipic Acid

A Hastelloy 300 ml Parr reactor was charged with degassed diglyme (50 ml), degassed deionised water (5.0 ml, 228 mmol), and degassed 3-pentenoic acid (15.0 ml, 148 mmol) under a stream of argon gas. A yellow solution of catalyst (0.2 mol % Pd relative to 3-pentenoic acid) consisting of palladium acetate (69.9 mg, 0.31 mmol), 1,2-bis[di(t-butyl)phosphinomethyl]benzene (236.6 mg, 0.60 mmol) and methane sulfonic acid (0.1 mL, 1.5 mmol) in diglyme (18 ml) was then injected into the reactor under a stream of argon gas. The Parr reactor was then pressurised with CO (60 bar). The reaction mixture was stirred at 1000 rpm. The Parr reactor was heated at 105° C. for 5 h. After 5 h, the reactor was cooled and vented and the mother liquor was transferred into a Schienk flask using a cannula under a stream of argon gas. The yellow mother liquor was placed in a refrigerator to further crystallise out the adipic acid. The reactor vessel was then opened to air. The obtained adipic acid was filtered, washed with acetonitrile and dried under vacuum at 60° C. to give 10.061 g of white adipic acid crystals (68.9 mmol). Analysis of the crude reaction mixture by ¹³C NMR showed the presence of 2-pentenoic acid and other by-products in addition to adipic acid.

### Example 11

### Carbonylation of Recycled Crude Reaction Mixture to Adipic Acid

A Hastelloy® 300 ml Parr reactor was charged with degassed 3-pentenoic acid (15.0 ml, 148 mmol), and the mother liquor from Example 10 under a stream of argon gas. The Parr reactor was then pressurised with CO (60 bar). The reaction mixture was stirred at 1000 rpm. The Parr reactor was heated at 105° C. for 5 h. After 5 h, the reactor was cooled and vented and the mother liquor was transferred into a Schienk flask using a cannula under a stream of argon gas. The yellow mother liquor was placed in a refrigerator to further crystallise out the adipic acid. The reactor vessel was then opened to air. The obtained adipic acid was filtered, washed with acetonitrile and dried under vacuum at 60° C. to give 11.243 g of white adipic acid crystals (76.9 mmol). Analysis of the crude reaction mixture by GC and NMR showed the presence of pentenoic acid isomers and γ-valerolactone in addition to adipic acid.

The processes described herein, and/or shown in the figures, are presented by way of example only and are not limiting as to the scope of the invention. Unless otherwise specifically stated, individual aspects of the processes may be modified, or may have been substituted therefore known equivalents, or as yet unknown substitutes such as may be developed in the future or such as may be found to be acceptable substitutes in the future. The processes may also be modified for a variety of applications while remaining within the scope and spirit of the claimed invention, since the range of potential applications is great, and since it is intended that the present processes be adaptable to many such variations.

1. A process for preparing a dicarboxylic acid comprising the steps:
(a) heating a lactone in the presence of a first catalyst system to produce an alkenoic acid; and
(b) contacting the alkenoic acid with carbon monoxide, water and a second catalyst system to produce a reaction composition comprising the second catalyst system and the dicarboxylic acid.

2. The process of claim 1, wherein step (a) is carried out substantially in the absence of water.

3. The process of claim 1 or 2, wherein the heating of the lactone in step (a) comprises reactive distillation, thereby providing a distillate comprising the alkenoic acid.

4. The process of any one of claims 1 to 3, wherein step (a) is carried out at a temperature at or above the normal boiling point of the lactone.

5. The process of any one of claims 1 to 4, wherein the first catalyst system comprises an acidic catalyst.

6. The process of any one of claims 1 to 5, wherein the first catalyst system comprises a heterogeneous solid catalyst.

7. The process of any one of claims 1 to 6, wherein the first catalyst system comprises a homogeneous catalyst.

8. The process of any one of claims 1 to 7, wherein the first catalyst system comprises one or more of alumina, silica, a zeolite, a clay, sulphuric acid, p-toluenesulfonic acid and methanesulfonic acid.

9. The process of any one of claims 1 to 8, wherein the first catalyst system comprises a mixture alumina and silica.

10. The process of any one of claims 1 to 9, wherein the alkenoic acid comprises a plurality of isomers.

11. The process of any one of claims 1 to 10, wherein step (b) is carried out substantially in the absence of oxygen.

12. The process of any one of claims 1 to 11, wherein step (b) is carried out at a temperature of between about 80° C. and about 120° C.

13. The process of any one of claims 1 to 12, wherein step (b) is carried out at a pressure of between about 3 bar and about 80 bar.

14. The process of any one of claims 1 to 13, wherein the second catalyst system comprises a palladium catalyst.

15. The process of claim 14, wherein the palladium catalyst has formula (I): 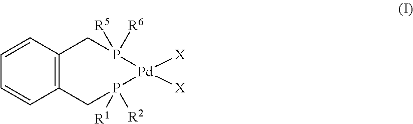
wherein X is a ligand and each of R¹, R², R⁵ and R⁶ is independently an optionally substituted organic group or R¹ and R² and/or R⁵ and R⁶, together with the P atom to which they are attached, form a cyclic group.

16. The process of any one of claims 1 to 15, wherein the second catalyst is prepared by combining a palladium compound, a bidentate diphosphine and an acid.

17. The process of claim 16, wherein the bidentate diphosphine has the formula (II):
(R¹)(R²)P—R³—Ar—R⁴—P(R⁵)(R⁶) (II)
wherein
Ar is an optionally substituted aromatic group;
R¹ and R² are either the same or different and represent a tertiary alkyl or, together with the P atom to which they are attached, form a phospha trioxa-adamantane group having formula (III): 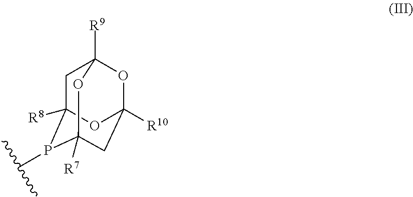
wherein R⁷, R⁸, R⁹ and R¹⁰ each independently represent an optionally substituted hydrocarbyl group;
R³ and R⁴ each independently represent an optionally substituted alkylene group; and
R⁵ and R⁶ each independently represent an optionally substituted organic group or together with the P atom to which they are attached, form a cyclic group.

18. The process of claim 16 or 17, wherein the palladium catalyst is prepared by combining palladium acetate, 1,2-bis[di(t-butyl)phosphinomethyl]benzene, and methane sulfonic acid.

19. The process of any one of claims 1 to 18, wherein a portion of the lactone is unreacted after step (a), the process further comprising the steps of:
(a1) separating part or substantially all of the unreacted lactone from the alkenoic acid; and
(a2) recycling part or substantially all of the separated unreacted lactone from step (a1) to step (a).

20. The process of claims 1 to 19, wherein a portion of the lactone is unreacted after step (a) and the reaction composition of step (b) comprises part or substantially all of the unreacted lactone from step (a), the process further comprising the steps of:
(b1) separating part or substantially all the unreacted lactone from the dicarboxylic acid; and
(b2) recycling part or substantially all of the separated unreacted lactone from step (b1) to step (a).

21. The process of any one of claims 1 to 20, wherein the reaction composition of step (b) comprises unreacted alkenoic acid, the process further comprising the steps of:
(b3) separating part or substantially all of the unreacted alkenoic acid from the dicarboxylic acid and
(b4) recycling part or substantially all of the separated unreacted alkenoic acid from step (b3) to step (a) and/or recycling part or substantially all of the separated unreacted alkenoic acid from step (b3) to step (b).

22. The process of any one of claims 1 to 21, further comprising the steps of:
(c) separating the dicarboxylic acid from the remainder of the reaction composition of step (b) to produce a first portion comprising the dicarboxylic acid and a second portion comprising the second catalyst system; and
(d) recycling the second portion, comprising the second catalyst system, to step (b).

23. The process of any one of claims 1 to 22, wherein the lactone is γ-valerolactone, whereby the alkenoic acid is pentenoic acid and the dicarboxylic acid is adipic acid.

24. The process of claim 23, wherein the pentenoic acid comprises one or more, or optionally all, of 2-pentenoic acid, 3-pentenoic acid and 4-pentenoic acid.

25. The process of claim 24, further comprising the step of preparing the γ-valerolactone by hydrogenation of levulinic acid.

26. The process of claim 25, further comprising the step of preparing the levulinic acid by acid catalysed hydrolysis of cellulose.

27. The process of claim 26, further comprising the step of preparing the cellulose by cracking lignocellulose.

28. A dicarboxylic acid prepared in accordance with the process of any one of claims 1 to 27.

29. The dicarboxylic acid of claim 28 having a purity of greater than about 99%.

30. The dicarboxylic acid of claim 28 or 29 wherein the dicarboxylic acid is adipic acid.

31. A method of preparing Nylon 6-6 comprising the step of copolymerising the adipic acid of claim 30 with hexamethylenediamine, thereby forming the Nylon 6-6.
